(19) United States
(12) Patent Application Publication
(10) Pub. No.: US 2021/0140962 A1
(43) Pub. Date: May 13, 2021

(54) COMPOUND AND METHOD OF MEASURING INTESTINAL PERMEABILITY AND LEAKY GUT

(71) Applicants: João Carlos Pinho da Silva, Paredes (PT); Fatima Patricia da Costa Oliveira Martins, Lisboa (PT)

(72) Inventors: João Carlos Pinho da Silva, Paredes (PT); Fatima Patricia da Costa Oliveira Martins, Lisboa (PT)

(21) Appl. No.: 16/487,720

(22) PCT Filed: Feb. 20, 2018
(86) PCT No.: PCT/IB2018/000212

§ 371 Date: Aug. 21, 2019

Related U.S. Application Data
(60) Provisional application No. 62/461,743, filed on Feb. 21, 2017.

(51) Int. Cl. G01N 33/566; G01N 33/551; A61K 33/00

(52) U.S. Cl. CPC G01N 33/566; A61K 33/00; G01N 33/551

(57) ABSTRACT

This invention provides a composition comprising:
a) an amount of a chromium-EDTA complex; and
b) an amount of riboflavin or an amount of glucose, or a mixture of riboflavin and glucose.

This application claims priority of U.S. Provisional Application No. 62/461,743, filed Feb. 21, 2017, the entire contents of which is hereby incorporated by reference herein.

Throughout this application various publications are referenced by numerical identifiers in parentheses. Full citations of these references can be found following the Examples. The disclosures of these publications in their entireties are hereby incorporated by reference into this application in order to more fully describe the state of the art to which this invention pertains.

### BACKGROUND OF THE INVENTION

The adult human intestinal barrier covers a surface of approximately 400 m² and requires approximately 40% of the body's energy supply. It prevents the loss of too much water and electrolytes, and also prevents the entry of antigens and microorganisms into the circulation while allowing exchange of molecules between host and environment for the absorption of essential nutrients from the diet. The intestinal barrier is a complex multilayer system, consisting of an external “physical” barrier and an internal “functional” immunological barrier. The interaction of these two barriers enables equilibrated intestinal permeability to be maintained. Intestinal permeability is a functional feature of the intestinal barrier closely linked to the gut microbiota and to elements of the intestinal mucosal immune system. Many factors can alter intestinal permeability such as gut microbiota modifications, mucus layer alterations, and epithelial damage, resulting in translocation of luminal content to the inner layers of the intestinal wall (Bischoff et al. 2014).

An individual suffering from increased gut permeability may be referred to as having ‘leaky gut syndrome’. Elevated leakage has been implicated in many human disorders with immunological components, including autoimmune diseases (Fasano, 2012), type 1 diabetes mellitus, obesity and type 2 diabetes mellitus (Horton et al. 2014), inflammatory bowel disease (e.g., Crohn's disease and ulcerative colitis) (Arrieta et al. 2006), colon cancer, celiac disease and irritable bowel syndrome (Konig et al. 2016; Arrieta et al. 2006), Parkinson's disease, environmental enteropathy and cancer (Resendez et al. 2015). Recent research shows that severe non-alcoholic steatohepatitis and non-alcoholic fatty liver disease are associated with increased intestinal permeability and indicates that an early phase of liver injury and inflammation contributes to this breach in the intestinal barrier (Luther et al. 2015).

The typical measurement of intestinal permeability in patients involves oral ingestion of probe molecules, which are not metabolized, but excreted in urine where they can be measured. (Camilleri et al. 2012). Some of the types of probes available to measure in vivo intestinal permeability are mannitol, disaccharides (e.g., lactulose, sucrose), or radioactive ⁵¹Cr-EDTA (Resendez et al. 2015; Camilleri et al. 2012). There are a number of intestinal permeability assays available through private healthcare, or supplied as home test kits from companies in the ‘Health and Wellness’ market (Genova Diagnostics, “Intestinal Permeability Assessment”). All of them present risks or problems, namely secondary effects, false negatives or false positives due to metabolism of the used compounds, reactivity with gut microbiota or internal tissues, low sensitivity and complexity of detection method, among others (Galipeau and Verdu, 2016).

### SUMMARY OF THE INVENTION

This invention provides a composition comprising:
a) an amount of a chromium-EDTA complex; and
b) an amount of riboflavin or an amount of glucose, or a mixture of riboflavin and glucose.

The invention also provides a method for measuring the level of exogenous chromium-EDTA complex in a sample obtained from a subject, comprising:
a) obtaining a sample from a subject;
b) removing free chromium from the sample; and
c) measuring the level of exogenous chromium-EDTA complex in the sample.

The invention also provides a method of identifying a subject as having elevated intestinal permeability, comprising:
a) administering an amount of chromium-EDTA complex to the subject,
b) measuring the level of an exogenous chromium-EDTA complex in a sample from the subject; and
c) identifying the subject as having elevated intestinal permeability if the non-radioactive chromium-EDTA complex in the subject's bodily fluid sample is elevated relative to a reference value.

The invention also provides a package comprising:
a) the composition of the invention; and
b) a storage device for the retention of a cumulative amount of the subject's sample over a time period.

### BRIEF DESCRIPTION OF THE DRAWINGS

FIG. 1. Proposed strategy for clinical usage of the intestinal permeability technology.

FIG. 2. Schematic methodology diagram for the Permeability technology.

FIG. 3. Structure of Chromium EDTA complex.

FIG. 4. Linearity and sensitivity of the analysis method.

FIG. 5. Intestinal Permeability proved significantly increased, with kit analysis, in an insulin resistance animal model.

FIG. 6. Demonstration of removal of background Cr ion from a urine sample using cation exchange resin.

### DETAILED DESCRIPTION OF THE INVENTION

This invention provides a composition comprising:
a) an amount of a chromium-EDTA complex; and
b) an amount of riboflavin or an amount of glucose, or a mixture of riboflavin and glucose.

In an embodiment, the chromium in the chromium-EDTA complex of the composition is a non-radioactive chromium isotope.

In another embodiment, the amount of chromium-EDTA complex in the composition is between 50-500 mg.

In another embodiment, the amount of riboflavin in the composition is between 1 mg and 150 mg. In another embodiment, the amount of riboflavin is between 50 mg and 100 mg. In another embodiment, the amount of riboflavin is between 60 mg and 90 mg. In another embodiment, the amount of riboflavin is between 70 mg and 80 mg. In another embodiment, the amount of riboflavin is 75 mg.

In another embodiment, the amount of glucose in the composition is between 1 g and 150 g. In another embodiment, the amount of glucose is between 25 g and 100 g. In another embodiment the amount of glucose is between 50 g and 75 g. In another embodiment, the amount of glucose is 75 g.

The invention also provides a method for measuring the level of exogenous chromium-EDTA complex in a sample obtained from a subject, comprising:
a) obtaining a sample from a subject;
b) removing free chromium from the sample; and
c) measuring the level of exogenous chromium-EDTA complex in the sample.

In an embodiment the level of exogenous chromium-EDTA complex is measured by mass spectrometry, Gas Furnace Atomic Absorption Spectroscopy (GFAAS), Electrothermal Atomic Absorption Spectrometry (ETAAS), Inductively Coupled Plasma Mass Spectrometry (ICP), or X-ray Fluorescence Spectrometry (XRF).

In an embodiment, the sample obtained from the subject is a bodily fluid sample.

In an embodiment, the bodily fluid sample is a urine sample.

In an embodiment, the method comprises administering an amount of chromium-EDTA complex to a subject before obtaining a sample from said subject.

In an embodiment, the chromium-EDTA complex is administered orally.

In an embodiment, the chromium in the chromium-EDTA complex is a non-radioactive isotope of chromium.

In an embodiment, the amount of chromium-EDTA complex is between 50 mg and 500 mg. In an embodiment, the amount of chromium-EDTA complex is between 350 mg.

In an embodiment, the method comprises collecting the subject's sample at a single time point after the chromium-EDTA complex is administered to the subject.

In an embodiment, the single time point is about 30 minutes, about 45 minutes, about 1 hour, about 1 hour and 30 minutes, about 2 hours, about 2 hours and 30 minutes, about 3 hours, about 4 hours, about 5 hours, about 6 hours, about 7 hours, about 8 hours, about 9 hours, about 10 hours or about 24 hours after the chromium-EDTA complex is administered to the subject.

In an embodiment, the method comprises administering an amount of riboflavin to the subject.

In an embodiment, riboflavin is administered at the same time as the amount of chromium-EDTA complex.

In an embodiment, the method includes normalizing the amount of chromium-EDTA complex in the subject's sample against the amount of riboflavin in the subject's sample.

In an embodiment, the amount of riboflavin in the composition is between 1 mg and 150 mg. In another embodiment, the amount of riboflavin is between 50 mg and 100 mg. In another embodiment, the amount of riboflavin is between 60 mg and 90 mg. In another embodiment, the amount of riboflavin is between 70 mg and 80 mg. In another embodiment, the amount of riboflavin is 75 mg.

In an embodiment, the amount of riboflavin in the subject's sample is determined by fluoroscopy.

In an embodiment, the method comprises measuring creatinine level in the subject's sample.

In an embodiment, the method comprises normalizing the amount of chromium-EDTA complex in the subject's sample against the amount of creatinine in the subject's sample.

In an embodiment, the method comprises administering an amount of glucose to the subject.

In an embodiment, the amount of glucose is administered orally.

In an embodiment, the amount of glucose is between 1 g and 150 g, preferably between 25 g and 100 g, preferably between 50 g and 75 g, or preferably about 75 g.

In an embodiment, the subject's blood is sampled:
a) at a baseline prior to administration of the glucose, non-radioactive chromium-EDTA complex and riboflavin; and
b) substantially concurrently with collection of the subject's urine sample.

In an embodiment, 2-20 μL of the subject's blood is sampled.

In an embodiment, the subject's plasma glucose level is measured using an electronic glucose meter.

In an embodiment, the method comprises incubating the subject's bodily fluid sample with an ion exchange resin.

In an embodiment, the ion exchange resin is a cation exchange resin.

In an embodiment, the cation exchange resin is an acidic cation exchange resin wherein the active group is nuclear sulfonic acid.

The invention also provides a method of identifying a subject as having elevated intestinal permeability, comprising:
a) administering an amount of chromium-EDTA complex to the subject,
b) measuring the level of an exogenous chromium-EDTA complex in a sample from the subject; and
c) identifying the subject as having elevated intestinal permeability if the non-radioactive chromium-EDTA complex in the subject's bodily fluid sample is elevated relative to a reference value.

In an embodiment, the reference value is based on the level of chromium-EDTA complex in a sample from a healthy control population.

In an embodiment, the reference value is based on the level of chromium-EDTA complex in the sample of a control population that does not have elevated intestinal permeability.

In an embodiment, the method is used to identify whether the subject is afflicted with autoimmune disease, type 1 diabetes mellitus, obesity, type 2 diabetes mellitus, inflammatory bowel disease, ulcerative colitis, Parkinson's disease, environmental enteropathy, cancer, food sensitivity, food allergy, irritable bowel syndrome, Crohn's disease, arthritis, celiac disease, or dermatological conditions such as eczema, psoriasis or acne.

In an embodiment, the chromium-EDTA complex is administered orally to the subject.

In an embodiment, the chromium in the chromium-EDTA complex is a non-radioactive isotope of chromium.

In an embodiment, the amount of chromium-EDTA complex is between 50 mg and 500 mg.

In an embodiment, the amount of chromium-EDTA complex is 350 mg.

The invention also provides a package comprising:
a) the composition of the invention; and
b) a storage device for the retention of a cumulative amount of the subject's sample over a time period.

In an embodiment, the composition is present in a single container for oral administration.

In an embodiment, the subject's sample is a bodily fluid sample.

In an embodiment, the bodily fluid sample is a urine sample.

Combinations of the above-described embodiments are also within the scope of the invention.

For the foregoing embodiments, each embodiment disclosed herein is contemplated as being applicable to each of the other disclosed embodiments. For instance, the elements recited in the method embodiments can be used in the composition and package embodiments described herein and vice versa.

### Normalization

The method measures the concentration of molecules other than non-radioactive Cr-EDTA in a subject's biological sample in order to establish an excretory ratio between the amount of Cr-EDTA found in the sample and a marker that is excreted by some other means than intestinal permeability (Elia et al. 1987). Two molecules used in the method for normalizing the amount of Cr-EDTA in a sample are riboflavin and creatinine (Resendez et al. 2014; Elia et al. 1987). For example, riboflavin and creatinine are markers used to measure an individual's urine excretion rate.

A normalization is performed by dividing the concentration of Cr-EDTA found in the sample with the concentration of the other marker known to be present in the sample. The use of a ratio over measurement of a single marker is that it removes the necessity of collecting the total volume of a subject's sample over a long period of time, (Elia et al. 1987). Instead, a ratio presents the opportunity to assess whether the subject's sample is indicative of an elevated Cr-EDTA concentration after taking a single sample at some predetermined time after administration of the Cr-EDTA complex to the subject.

### Definitions

As used herein “chromium EDTA complex” has the chemical formula, C10H13CrN2O8 and a molecular weight of 341.21 g/mol. The chemical structure is shown in FIG. 3.

As used herein “non-radioactive chromium isotope” means any one of ⁵²Cr, ⁵³Cr, or ⁵⁴Cr.

It is understood that where a parameter range is provided, all integers within that range, tenths thereof, and hundredths thereof, are also provided by the invention. For example, “0.2-5 mg” is a disclosure of 0.2 mg, 0.21 mg, 0.22 mg, 0.23 mg etc. up to 0.3 mg, 0.31 mg, 0.32 mg, 0.33 mg etc. up to 0.4 mg, 0.5 mg, 0.6 mg etc. up to 5.0 mg.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1: Preparation of Non-Radioactive Cr-EDTA

The non-radioactive chromium EDTA complex serving as a tracer molecule is shown as (FIG. 3) which has the chemical formula, C10H13CrN2O8 and a molecular weight of 341.21 g/mol (“Chromium EDTA Complex” PubChem Compound Database; CID=28282). This complex is synthesized as follows:

1) Dissolve 67 mg of the non-radioactive CrCl₃.6H₂O in 1 ml of water.
2) Dissolve 100 mg of disodium EDTA in 2 ml of water.
3) The two solutions (CrCl₃.6H₂O and disodium EDTA) are combined and heated to 100° C. for 1 hour, and then cooled.
4) The cooled solution is passed through an amount of strongly acidic cation exchange resin wherein the active group is nuclear sulfonic acid thereby scavenging any free Cr ions from the aqueous solution.

The compound is synthesized as an aqueous solution with the concentration of about 2.5 mM (0.86 g/L), is non-toxic (i.e. is comprised of materials that have been verified as being non-toxic by the FDA.

### Example 2

### a) Measurement of Intestinal Permeability by Non-Radioactive Cr-EDTA Administration and Urine Analysis Performed in Animals

The following is an example of non-radioactive Cr-EDTA administration and urine analysis being performed in an animal study. 100 μg of non-radioactive Cr-EDTA in aqueous solution was administered to C57BL/6 mice and Wistar rats by oral gavage. A urine sample from the animals was then collected 6-10 hours after administration.

The urine sample taken from the animal was incubated with strongly acidic cation exchange resin wherein the active group is nuclear sulfonic acid, e.g., 33% by volume slurry of DOWEX-50-H+® cation exchange resin equilibrated with distilled water at room temperature.

Naturally occurring background Cr ions in the urine were correspondingly bound to the resin. Removal of free Cr ions from a urine sample by treatment with cation exchange resin is demonstrated in FIG. 6. The samples were then centrifuged, with the supernatant containing the fraction of administered Cr-EDTA that crossed the intestinal barrier.

The supernatant was injected directly into a Gas Furnace Atomic Absorption Spectroscope (GFAAS) using a graphite tube. The GFAAS apparatus had been calibrated to yield the concentration of Cr in each sample.

b) In Vivo Studies of Measuring Intestinal Permeability in Animals with Insulin Resistance.

The method of Example 2(a) was tested in a rodent model which was induced to experience a gradual deterioration of insulin sensitivity and eventual onset of diabetes, which has been shown to increase intestinal permeability (Horton, 2014). The rodent models experienced degraded intestinal permeability over time which corresponded with a measurable increase in the ratio of Cr to creatinine in urine samples over the same period. This experiment demonstrated a successful measurement of increasing intestinal permeability over time with the progressive onset of disease using the method described in Example 2a (FIG. 5).

### Example 3: In Vitro Determination of Linearity and Sensitivity of Quantifying Non-Radioactive Cr-EDTA in Urine

The following is an example of a determination that the non-radioactive Cr-EDTA quantification methods used herein were sensitive to differing concentrations of Cr-EDTA in a urine sample and responded in a linear manner. A range of standards of known concentrations of chromium in solution were prepared, and the absorbance of each solution was measured at 357.9 nm. Samples of urine were then spiked with Cr-EDTA to produce the same concentration as the standards, and the absorbance of the solution was again measured at 357.9 nm. Comparison between the curve of standards and the curve of Cr-EDTA in urine produced similar curves, demonstrating the linearity of the measurement method (FIG. 4).

### Example 4: Normalization of Cr-EDTA Using Riboflavin

Riboflavin is absorbed through the intestine via paracellular intestinal transport, which is an independent mechanism than Cr-EDTA passing through the intestinal walls. Amounts of riboflavin in the urine are therefore a standard of normal intestinal absorption rates, independent of the level of intestinal permeability. The ratio of the concentrations of Cr-EDTA and riboflavin in a urine sample is therefore considered a measure of intestinal permeability normalized against a separate, independent means of intestinal uptake. (Resendez et al. 2015).

The primary advantage gained by comparing Cr-EDTA and riboflavin is that it affords a shortened time period over which urine must be collected. Once there are sufficient amounts of Cr-EDTA and riboflavin in a urine sample, a measure of intestinal permeability can be made, as opposed to having to collect urine over many hours to capture how long it takes to recover a dose of Cr-EDTA.

The method for the quantification of riboflavin is as described by WIPO International Publication Number WO 2016/036887 A1, (“Singaram et al.”) which is incorporated herein by reference. Singaram et al. describes a method in which riboflavin is ingested by a patient, and the riboflavin in the patient's urine sample is assayed by autofluorescence. Singaram et al. also describes a method in which fluorescence measurement of the amount of sugar in the biological sample using organoborane compound coupled to a fluorophore is normalized against riboflavin fluorescence in the same sample.

The level of non-radioactive Cr-EDTA is measured in mice administered with non-radioactive 100 μg of Cr-EDTA and an amount of riboflavin by normalizing the amount of Cr-EDTA in the urine sample against riboflavin in the same sample, wherein the amount of Cr-EDTA is measured in the urine sample using the methods presented herein and the amount of riboflavin is measured by fluorescence.

### Example 5: Normalization of Cr-EDTA Using Creatinine

Creatinine levels in a subject are measured as a means to establish a subject's glomerular filtration rate (GFR). A GFR measures the flow rate of filtered fluid through the kidney, indicating the quality of the subject's kidney function. Creatinine levels are measured because creatinine clearance (the volume of blood plasma that is cleared of creatinine per unit of time) is a good approximation of GFR. (Stevens et al. 2006).

Creatinine levels are measured using a kit. For example, Creatinine Assay Kit (Enzymatic), available at crystalchem.com/creatinine-assay-kit.html.

Establishing a subject's GFR allows the sampling of Cr-EDTA at a single time point, rather than the standard measurement of 6 and 24-hour cumulative amounts of urinary production. A normalization is done by taking the amount of excreted Cr-EDTA in the urine sample, and comparing it against the amount of creatinine in the subject's urine sample, the proportion being a measure of gut permeability.

The level of non-radioactive Cr-EDTA is measured in mice administered with non-radioactive 100 μg of Cr-EDTA by normalizing the amount of Cr-EDTA in the urine sample against creatinine in the same sample, wherein the amount of Cr-EDTA is measured in the urine sample using the methods presented herein and the amount of creatinine is measured using a kit.

### Example 6: Measuring Intestinal Permeability by Non-Radioactive Cr-EDTA Administration and Urine Analysis Performed in Humans

Dosages of the Cr-EDTA and Riboflavin complex are ingested orally and a baseline sample of urine is collected at that time. The dosage of Cr-EDTA administered is 100 mg and the dosage of Riboflavin is 50 mg. The patients need not fast for any length of time during utilization of this method, and may also eat or drink throughout as needed. For a period of 6 hours, the volume of each urine voiding is measured and a sample is retained for analysis.

Between 1-2 mL of each urine sample is then incubated with a 33% by volume slurry of DOWEX-50-H+ cation exchange resin, which has been equilibrated in distilled water. The resin/urine solution is then centrifuged at 4 C and 3000 RCF for 20 minutes.

The supernatant is separated from the resin with a pipette and 20 μL of the supernatant sample is then injected into a GFAAS which has been calibrated to provide accurate concentrations of Cr in the sample.

Simultaneously, 100 μL of the supernatant is diluted into 900 μL of 95% EtOH, vortexed, and centrifuged. 40 μL of the supernatant is pipetted into a well on a plate, and fluorescence at 450/580 nm is read on a plate reader. The concentration of riboflavin is calculated in mg/mL.

Intestinal permeability is then determined by comparing the concentrations of Cr-EDTA and riboflavin in the urine samples.

### REFERENCES

Arrieta et al., Alterations in Intestinal Permeability. Gut. 2006; 55(10): 1512-1520.
Bischoff et al., Intestinal Permeability—A New Target For Disease Prevention And Therapy, BMC Gastroenterology 2014; 14:189.
Camilleri et al., Intestinal Barrier Function in Health and Gastrointestinal Disease, Neurogastroenterology & Motility Vol 24, p. 503-512, 2012
Elia et al., Evaluation of Mannitol, Lactulose and 51Cr-Labelled Ethylenediaminetetra-acetate as Markers of Intestinal Permeability in Man. Clinical Science. 1987; 73, 197-204.
Fasano, Leaky Gut and Autoimmune Diseases, Clin. Rev. Allergy & Immunol. 2012; 42(1):71-78.
Galipeau, and Verdu, The complex task of measuring intestinal permeability in basic and clinical science. Neurogastroenterol Motil, 2016; 28(7), p. 879-83
Genova Diagnostics “Intestinal Permeability Assessment” [online], [retrieved on 2016-10-18]. Retrieved from the Internet:<www.gdx.net/product/intestinal-permeability-assessment-urine>.
Horton et al., Increased Intestinal Permeability to Oral Chromium (⁵¹Cr)-EDTA in human Type 2 diabetes, Diabetic Medicine. 2014; 31, 559-563.
Konig, et al., Human Intestinal Barrier Function in Health and Disease. Clin. Transl. Gastroenterol, 2016; 7(10), p. 196 Luther et al., Hepatic Injury in Nonalcoholic Steatohepatitis Contributes to Altered Intestinal Permeability. CMGH. 2015; 1(2):222-232.
National Center for Biotechnology Information. “Chromium EDTA Complex” PubChem Compound Database; CID=28282, Retrieved from the Internet: pubchem.ncbi.nlm.nih.gov/compound/28282 [retrieved Oct. 19, 2016].
Resendez et al., Rapid Small Intestinal Permeability Assay Based On Riboflavin and Lactulose Detected by Bis-boronic Acid Appended Benzyl Viologens, Clinica Chimica Acta. 2015; 439: 115-121.
Resendez, et al., Multiwell Assay for the Analysis of Sugar Gut Permeability Markers: Discrimination of Sugar Alcohols with a Fluorescent Probe Array Based on Boronic Acid Appended Viologens. Anal. Chem, 2016; 88(10), p. 5444-52.
Stevens LA, Coresh J, Greene T, Levey AS (June 2006). “Assessing kidney function—measured and estimated glomerular filtration rate”. The New England Journal of Medicine. 354 (23): 2473-83.

What is claimed is:

1. A composition comprising:
a) an amount of a chromium-EDTA complex; and
b) an amount of riboflavin or an amount of glucose, or a mixture of riboflavin and glucose.

2. The composition of claim 1, wherein the chromium in the chromium-EDTA complex is a non-radioactive chromium isotope.

3. The composition according to claim 1 or 2, wherein the amount of chromium-EDTA complex is between 50-500 mg.

4. The composition according to claim 1 or 2, wherein the amount of riboflavin is between 1 mg and 150 mg, preferably between 50 mg and 100 mg, more preferably between 60 mg and 90 mg, more preferably between 70 mg and 80 mg, or more preferably about 75 mg.

5. The composition according to any one of claims 1-3, wherein the amount of glucose is between 1 g and 150 g, preferably between 25 g and 100 g, preferably between 50 g and 75 g, or preferably about 75 g.

6. A method for measuring the level of exogenous chromium-EDTA complex in a sample obtained from a subject, comprising:
a) obtaining a sample from a subject;
b) removing free chromium from the sample; and
c) measuring the level of exogenous chromium-EDTA complex in the sample.

7. The method of claim 6, wherein the level of exogenous chromium-EDTA complex is measured by mass spectrometry, Gas Furnace Atomic Absorption Spectroscopy (GFAAS), Electrothermal Atomic Absorption Spectrometry (ETAAS), Inductively Coupled Plasma Mass Spectrometry (ICP), or X-ray Fluorescence Spectrometry (XRF).

8. The method of claim 6 or 7, wherein the sample obtained from a subject is a bodily fluid sample.

9. The method of claim 8, wherein the bodily fluid sample is a urine sample.

10. The method of any one of claims 6-9, further comprising a step of administering an amount of chromium-EDTA complex to the subject before step (a).

11. The method of claim 10, wherein the chromium-EDTA complex is administered orally.

12. The method of claim 10 or claim 11, wherein the chromium in the chromium-EDTA complex is a non-radioactive isotope of chromium.

13. The method of any one of claims 10-12 wherein the amount of chromium-EDTA complex is between 50 mg and 500 mg.

14. The method of any one of claims 10-13 wherein the amount of chromium-EDTA complex is 350 mg.

15. The method of any one of claims 6-14, wherein step (a) comprises collecting the subject's sample at a single time point after the chromium-EDTA complex is administered to the subject.

16. The method of claim 15, wherein the single time point is about 30 minutes, about 45 minutes, about 1 hour, about 1 hour and 30 minutes, about 2 hours, about 2 hours and 30 minutes, about 3 hours, about 4 hours, about 5 hours, about 6 hours, about 7 hours, about 8 hours, about 9 hours, about 10 hours or about 24 hours after the chromium-EDTA complex is administered to the subject.

17. The method of any one of claims 6-16, further comprising administering an amount of riboflavin to the subject.

18. The method of claim 17, wherein riboflavin is administered at the same time as the amount of chromium-EDTA complex.

19. The method of claim 17 or claim 18, further comprising normalizing the amount of chromium-EDTA complex in the subject's sample against the amount of riboflavin in the subject's sample.

20. The method of claim any one of claims 17-19, wherein the amount of riboflavin is between 1 mg and 150 mg, preferably between 50 mg and 100 mg, more preferably between 60 mg and 90 mg, more preferably between 70 mg and 80 mg, or more preferably about 75 mg.

21. The method of claim any one of claims 17-20, wherein the amount of riboflavin is 75 mg.

22. The method of any one of claims 17-21, wherein the amount of riboflavin in the subject's sample is determined by fluoroscopy.

23. The method of any one of claims 6-22, further comprising measuring creatinine level in the subject's sample.

24. The method of claim 23, further comprising normalizing the amount of chromium-EDTA complex in the subject's sample against the amount of creatinine in the subject's sample.

25. The method of any one of claims 6-24 further comprising administering an amount of glucose to the subject.

26. The method of claim 25, wherein an amount of glucose is administered orally.

27. The method of claim 25 or 26, wherein the amount of glucose is between 1 g and 150 g, preferably between 25 g and 100 g, preferably between 50 g and 75 g, or preferably about 75 g.

28. The method of any one of claims 25-27, wherein the subject's blood is sampled:
a) at a baseline prior to administration of the glucose, non-radioactive chromium-EDTA complex and riboflavin; and
b) substantially concurrently with collection of the subject's urine sample.

29. The method of claim 28, wherein 2-20 μL of the subject's blood is sampled.

30. The method of claim 28 or 29 wherein the subject's plasma glucose level is measured using an electronic glucose meter.

31. The method of any one of claims 6-28, wherein step (b) comprises incubating the subject's bodily fluid sample with an ion exchange resin.

32. The method of claim 29, wherein the ion exchange resin is a cation exchange resin.

33. The method of claim 30, wherein the cation exchange resin is an acidic cation exchange resin wherein the active group is nuclear sulfonic acid.

34. A method of identifying a subject as having elevated intestinal permeability, comprising:
a) administering an amount of chromium-EDTA complex to the subject;
b) measuring the level of an exogenous chromium-EDTA complex in a sample from the subject according to any one of claims 6-33; and
c) identifying the subject as having elevated intestinal permeability if the non-radioactive chromium-EDTA complex in the subject's bodily fluid sample is elevated relative to a reference value.

35. The method of claim 34, wherein the reference value is based on the level of chromium-EDTA complex in a sample from a healthy control population.

36. The method of claim 34, wherein the reference value is based on the level of chromium-EDTA complex in the sample of a control population that does not have elevated intestinal permeability.

37. The method of any one of claims 34-36 for identifying whether the subject is afflicted with autoimmune disease, type 1 diabetes mellitus, obesity, type 2 diabetes mellitus, inflammatory bowel disease, ulcerative colitis, Parkinson's disease, environmental enteropathy, cancer, food sensitivity, food allergy, irritable bowel syndrome, Crohn's disease, arthritis, celiac disease, or dermatological conditions such as eczema, psoriasis or acne.

38. The method of any one of claims 34-37, wherein the chromium-EDTA complex is administered orally to the subject.

39. The method of any one of claims 34-38, wherein the chromium in the chromium-EDTA complex is a non-radioactive isotope of chromium.

40. The method of any one of claims 34-38, wherein the amount of chromium-EDTA complex is between 50 mg and 500 mg.

41. The method of claim 40, wherein the amount of chromium-EDTA complex is 350 mg.

42. A package comprising:
a) the composition of any one of claims 1-5; and
b) a storage device for the retention of a cumulative amount of a subject's sample over a time period.

43. The package of claim 42, wherein the composition is present in a single container for oral administration.

44. The package of claim 42 or claim 43, wherein the subject's sample is a bodily fluid sample.

45. The package of claim 44, wherein the bodily fluid sample is a urine sample.
